(19) United States
(12) Patent Application Publication
(10) Pub. No.: US 2020/0077997 A1
(43) Pub. Date: Mar. 12, 2020

(54) RADIOLUCENT RETRACTOR

(71) Applicants: Eric McCormick, Noblesville, IN (US); Mitchell Logan McCormick, Noblesville, IN (US)

(72) Inventors: Eric McCormick, Noblesville, IN (US); Mitchell Logan McCormick, Noblesville, IN (US)

(21) Appl. No.: 16/126,177

(22) Filed: Sep. 10, 2018

(51) Int. Cl. A61B 17/02

(52) U.S. Cl. CPC A61B 17/0206; A61B 2017/0023

(57) ABSTRACT

A generally wishbone-shaped disposable radiolucent retractor, including a transverse portion, a first leg extending from the transverse portion, a second leg extending from the transverse portion, wherein the first and second legs define a plane, a first plate operationally connected to the first leg and spaced from the transverse portion, and a second plate operationally connected to the second leg and spaced from the transverse portion. The first and second leg define and angle of about 35 degrees. The retractor is made of a generally electrically insulating nonmagnetic radiolucent material, such as nonporous medical grade thermoplastic resin.

### BACKGROUND

During surgery, the surgeon must be able to both see what he is doing as well as to have sufficient room to maneuver. Retractors are devices commonly used to pull tissue away from the operative area to allow the surgeon room and visibility. Retractor devices allow access to otherwise concealed tissues and surrounding areas, and are useful in both general surgery as well as microsurgical procedures.

Most retractors are made of metal, allowing for multiple uses and are typically disinfected by means of autoclaving. However, metal retractors have the disadvantage of being radiopaque. In many microsurgical procedures, there is insufficient room available for the surgeon to completely visually observe the operative area, especially from all relevant angles, and x-ray or like penetrating imaging is used in real time to guide the surgeon. Such imaging is difficult to perform when multiple surgical tools, including stationary metal retractors, are in use during the operation.

Another drawback of most retractors is that they must be manually held in place during the operation. Such requisite manpower adds to the cost of surgery as well as crowding the operating venue.

Thus, there remains a need for an improved retractor. The present invention addresses this need.

### BRIEF DESCRIPTION OF THE DRAWINGS

FIG. 1 is a first perspective view of a retractor device according to a first embodiment of the novel technology.

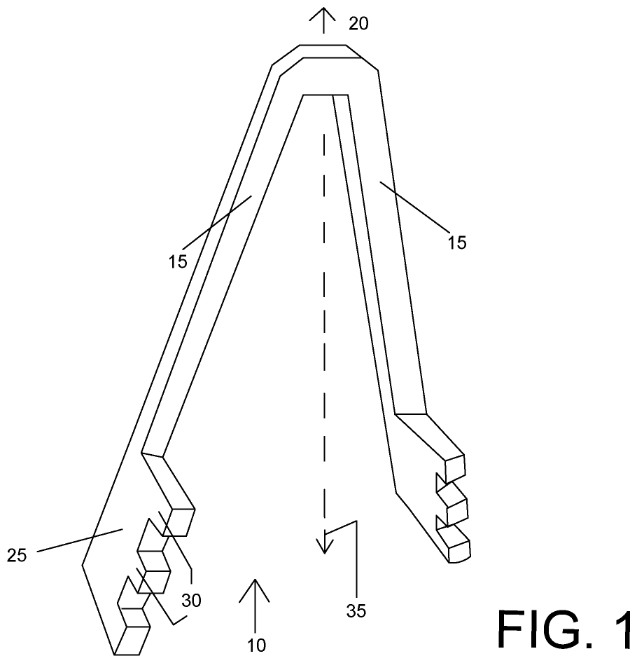

FIG. 2 is a second perspective view of the embodiment of FIG. 1.

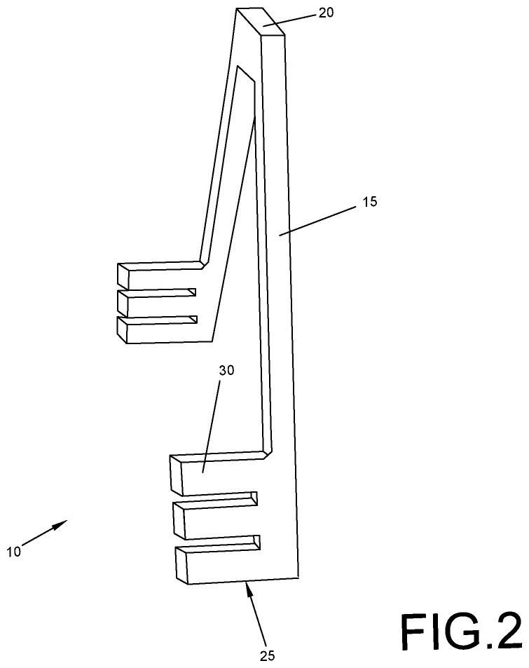

FIG. 3 is a third perspective side view of the embodiment of FIG. 1.

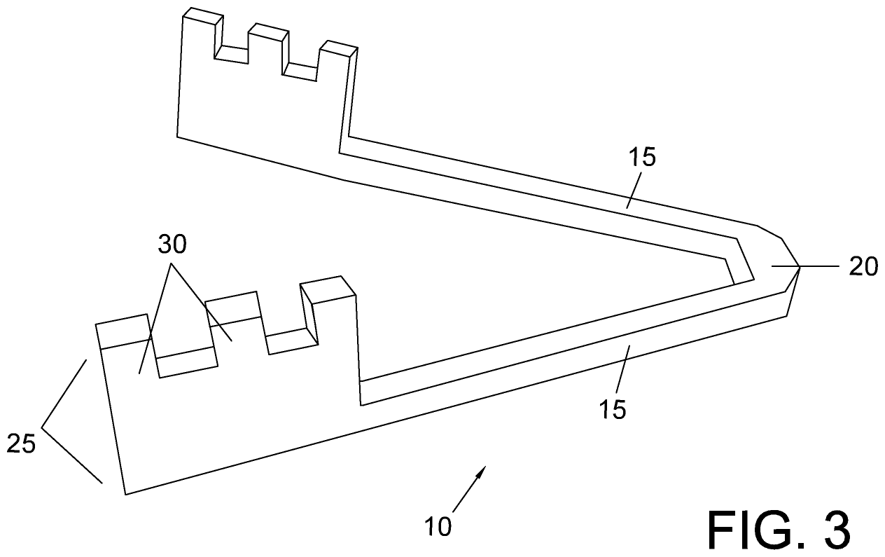

FIG. 4 is a fourth perspective view of the embodiment of FIG. 1 and including a locking device.

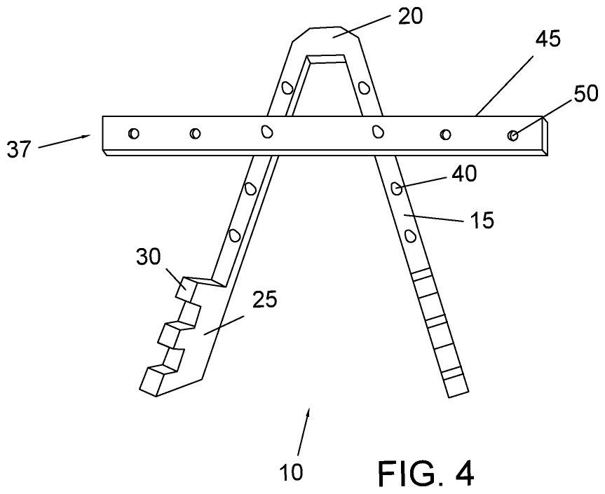

FIG. 5 is a perspective view of the embodiment of FIG. 1 but with curved fingers.

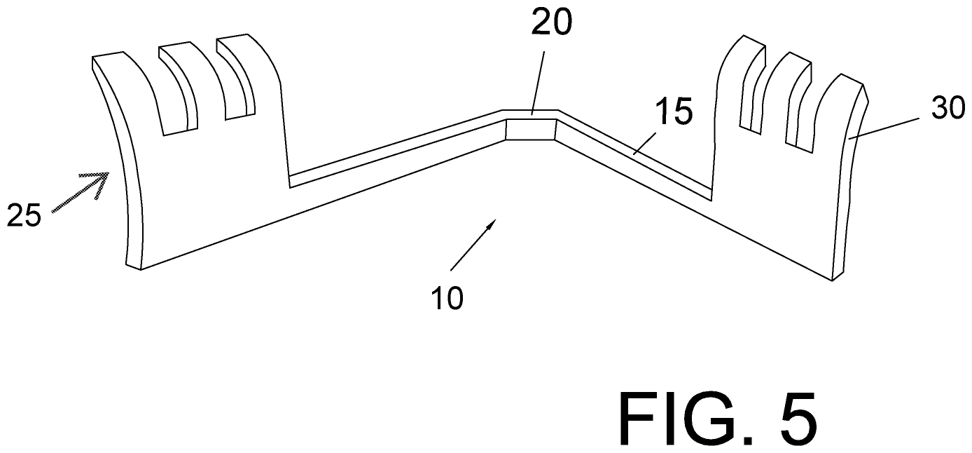

FIG. 6 is a top plan view of a second embodiment of the present novel technology, a surgical kit including the embodiment of FIG. 1 embodiment of FIG. 1.

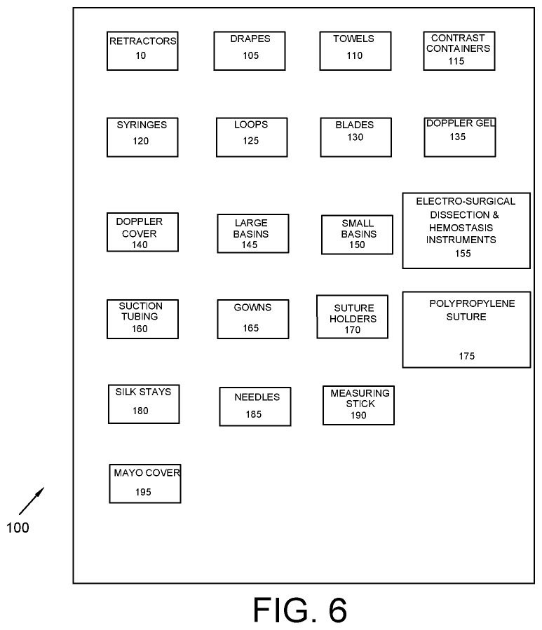

### DETAILED DESCRIPTION

For the purposes of promoting an understanding of the principles of the novel technology and presenting its currently understood best mode of operation, reference will now be made to the embodiments illustrated in the drawings and specific language will be used to describe the same. It will nevertheless be understood that no limitation of the scope of the novel technology is thereby intended, with such alterations and further modifications in the illustrated device and such further applications of the principles of the novel technology as illustrated therein being contemplated as would normally occur to one skilled in the art to which the novel technology relates.

The present novel technology relates to a single use or ‘disposable’ device 10 for holding tissues away from a field of operation, such as by pushing open the edges of a wound to allow access thereinto. The device 10 is electrically insulating, non-magnetic, and radiotransparent, and is typically a unitary, homogeneous body molded or printed from a (typically) medical grade thermoplastic resin or like material, and is typically nonporous. The device includes a pair of elongated legs 15 extending from a transverse portion 20 to define a generally wishbone-shaped retractor 10. A tissue-engaging plate portion 25 is typically positioned at the terminal end (opposite the transverse portion 20) of each leg 15 for engaging and urging tissue away from a wound or incision. The plate 25 may further include a plurality of tissue-gripping fingers 30 extending away from the leg 15. The fingers 30 may be straight or curved to better engage the tissue. Curved fingers 30 typically curve away from the retractor 10. In some embodiments, the fingers 30 may curl into hooks to even better grip tissue.

In one embodiment, each leg is about 8 cm. in length and has a generally square cross section of 8 mm. on a side. The transverse portion is about 1.5 cm. long. The legs 15 are not parallel, and typically each leg 15 extends away from the other leg 15 with the plates 25 being spaced about 6 cm. away from one another, thus defining an angle between fifteen and sixty degrees, more typically between thirty and forty-five degrees, and still more typically about thirty-five degrees. In other words, each leg 15 extends away from a major axis 35, defined as bisecting the transverse portion 20 and lying in the same plane as the legs 15, at an angle of about seventeen and a half degrees. When pressed together, the legs 15 typically exert a force of about between about twenty-five and about fifty Newtons, more typically about thirty-five Newtons, away from one another, with each leg typically exerting a force of about between about ten and twenty Newtons, more typically about fifteen Newtons, against the edge of the wound or incision. The device 10 thus is described by a spring constant of between about 400 and about 850 N/m, more typically about 600 N/m.

The retractor 10 functions as an elongated transverse leaf spring, insofar as the legs 15, when pushed together, exert an urging force to return them to their original resting orientation. The force exerted by the legs 15 may be varied by varying the thickness of the legs 15 and transverse piece 20, the length of the legs 15, the thickness of the legs 15, the angle defined by the legs 15, and/or the composition of the retractor 10.

Prior to use, each retractor 10 is typically sterilized, such as via immersion in an ethylene oxide bath.

In some embodiments, the legs 15 each include a locking device 37 for preventing the legs 15 from moving relative to one another. One exemplary locking device 37 includes a plurality of spaced raised nubs or bullets 40 positioned on each leg 15, that cooperate with an elongated locking strip or latch 45 having a plurality of spaced apertures formed therethrough 50, each aperture 50 sized to snugly engage a bullet 40. The locking strip 45 may be engaged to a bullet 40 extending from each leg 15 to secure the retractor 10 once it is engaged with a patient to prevent unwanted expansion or constriction of the retractor legs 15 to control and maintain the dimensions of an incision opening.

In some embodiments, the retractor 10 is included as part of a kit 100. Kit 100 typically includes at least one or more (typically a plurality) of each of the following: retractors 10, drapes 105, towels 110, contrast containers 115, syringes 120, and loops 125. Each kit 100 may also include one or more of the following: blade (typically no. 20) 130, Doppler gel 135, Doppler cover 140, large basin 145, small basin 150, electrosurgical dissection and hemostasis instrument 155, suction tubing 160, gown 165, suture holder 170, 6-0 polypropylene suture 175, 2-0 silk stays 180, needles 185, measuring stick 190, and/or mayo cover 195.

In operation, the legs 15 are first pressed together and are inserted far enough into the wound or incision such that the plates 25 and/or fingers 30 engage tissue on either side of the wound. The pressure holding the legs 15 together is then released to allow the legs 15 to exert a spring force against the tissue, urging the wound open. The retractor 10 may then be left in place to hold the wound open, and may optionally be locked by engaging the strip 45 with bullets 40 on either leg 15. Once the wound is no longer desired to be held open, the legs 15 are once again urged toward one another and the retractor 10 is removed from the wound. X-rays made during the procedure will not be impeded or obstructed by the plastic retractor, making the retractor attractive for procedures on the carotid, thrombectomies, embolectomies, pacemaker implantation, and the like.

While the novel technology has been illustrated and described in detail in the drawings and foregoing description, the same is to be considered as illustrative and not restrictive in character. It is understood that the embodiments have been shown and described in the foregoing specification in satisfaction of the best mode and enablement requirements. It is understood that one of ordinary skill in the art could readily make a nigh-infinite number of insubstantial changes and modifications to the above-described embodiments and that it would be impractical to attempt to describe all such embodiment variations in the present specification. Accordingly, it is understood that all changes and modifications that come within the spirit of the novel technology are desired to be protected.

1) A disposable device for tissue retraction, comprising:
an elongated transverse portion having a first end and a second end;
a first leg extending from the first end of the transverse portion;
a second leg extending from the second end of the transverse portion;
a first plate operationally connected to the first leg and spaced from the transverse portion; and
a second plate operationally connected to the second leg and spaced from the transverse portion;
wherein the first and second leg define and angle of about 35 degrees;
wherein retractor is a unitary, homogeneous body made of a generally electrically insulating nonmagnetic radiolucent material; and
wherein a force of about 35 Newtons is required to urge the first plate into contact with the second plate.

2) The device of claim 1, wherein the retractor is made of nonporous medical grade thermoplastic resin.

3) The device of claim 1, wherein each leg is about 8 centimeters long.

4) The device of claim 1, wherein a plurality of fingers extend from each respective plate.

5) The device in claim 4 wherein each finger is curved away from the retractor.

6) The device of claim 1, and further comprising a plurality of spaced, raised nubs extending from each respective leg; and an elongated locking strip having a plurality of apertures, each aperture sized to snugly receive a raised nub.

7) A method for retracting tissue comprising:
a) moving first and second ends of respective first and second legs of a thermoplastic resin retractor together;
b) inserting the first and second ends into an incision;
c) allowing the first and second ends to exert an urging force of between 10 and 20 Newtons on the incision;
d) removing the retractor from the incision; and
e) discarding the retractor.

8) The method of claim 7 and further comprising:
f) after c) and before d), x-ray imaging through the retractor.

9) The method of claim 7 and further comprising:
g) after c) and before d), locking the legs of the retractor in place with a radiolucent latch.

10) The method of claim 7 and further comprising:
g) before a), disinfecting the retractor with ethylene oxide.

11) The method of claim 7 wherein the urging force is about 15 Newtons.

12) A generally wishbone-shaped disposable radiolucent retractor, comprising:
a leaf spring, further comprising:
a transverse portion;
a first leg extending from the transverse portion; and
a second leg extending from the transverse portion, wherein the first and second legs define a plane;
a first plate operationally connected to the first leg and spaced from the transverse portion; and
a second plate operationally connected to the second leg and spaced from the transverse portion;
wherein the first and second leg define and angle of about 35 degrees;
wherein retractor is made of a generally electrically insulating nonmagnetic radiolucent material; and
wherein the leaf spring and the respective plates define a unitary, homogeneous body; and
wherein the leaf spring has a spring constant between 400 and 850 N/m.

13) The device of claim 12, wherein the retractor is made of nonporous medical grade thermoplastic resin.

14) The device of claim 12, wherein each leg is about 8 centimeters long.

15) The device of claim 12, wherein a plurality of fingers extend from each respective plate and wherein each finger extends perpendicularly to the plane.

16) The device in claim 12 wherein a plurality of fingers extend from each respective plate and wherein each finger is curved away from the retractor.

17) The device of claim 12, and further comprising a plurality of spaced, raised nubs extending from each respective leg; and an elongated locking strip having a plurality of apertures, each aperture sized to snugly receive a raised nub.

18) The device of claim 12 wherein the spring constant is 600 N/m.
